(19) United States
(12) Patent Application Publication
(10) Pub. No.: US 2024/0358926 A1
(43) Pub. Date: Oct. 31, 2024

(54) Expulsion assembly of a medicament delivery device

(71) Applicant: SHL Medical AG, Zug (CH)

(72) Inventors: Ming-Ting Yin, Taoyuan City (TW); Philipp Zingel, Taoyuan City (TW); Roman Käslin, Zurich (CH); Lino Manuel Heckhorn Ghilardi, Zurich (CH)

(21) Appl. No.: 18/293,981

(22) PCT Filed: Aug. 3, 2022
(86) PCT No.: PCT/EP2022/071832

§ 371 Date: Jan. 31, 2024

(30) Foreign Application Priority Data
Aug. 9, 2021 (EP) 21190459.4

(51) Int. Cl. A61M 5/28; A61M 5/32

(52) U.S. Cl. CPC A61M 5/281; A61M 5/3243; A61M 2207/10

(57) ABSTRACT

An expulsion assembly (100) of a medicament delivery device (102) for expelling medicament from a medicament container structure (250), the expulsion assembly comprising a container holder (125) having a container proximal end (212) and a container distal end (214), the container holder being configured to house the medicament container structure, and an axially extending needle cover (123), the needle cover having a needle cover proximal end (202) comprising a tubular shaped needle cover portion (206), and a needle cover distal end (204), wherein the container holder is at least partly enclosed by the needle cover, wherein a portion (121) of the outer surface of the container holder (125) and a portion (129) of the needle cover form an interface section (127) comprising an axially extending groove (220) and a protrusion (222) in which the container holder and the needle cover are arranged in abutment with each other.

### CROSS-REFERENCE TO RELATED APPLICATIONS

The present application is a U.S. National Phase Application pursuant to 35 U.S.C. § 371 of International Application No. PCT/EP2022/071833 filed Aug. 3, 2022, which claims priority to EP Patent Application No. 21190459.4 filed Aug. 9, 2021. The entire disclosure contents of these applications are herewith incorporated by reference into the present application.

### TECHNICAL FIELD

The present disclosure generally relates to an expulsion assembly of a medicament delivery device for expelling medicament from a flexible medicament container, as well as a medicament delivery device comprising such expulsion assembly and a method of assembling an expulsion assembly.

### BACKGROUND

Medicament is conventionally expelled from a medicament delivery device to a so-called dose delivery site, e.g. through the skin of a person in need of the medicament. The medicament delivery device thus comprises a medicament container that contains the medicament. The medicament container is preferably arranged within a housing and is expelled when exposed to activation from e.g. a drive assembly.

These days, a number of different medicament delivery devices exist, including various types of pen injectors, autoinjectors and on-body devices.

Although many of these devices have enabled major improvements in the management of a number of medical conditions, various limitations do still exist in the current technology. Not least amongst these are the robustness and mechanical properties of the medicament delivery device. In particular, the expulsion assembly forms an issue of further improvement. In considering these problems, the applicant has appreciated that various developments could be made to help improve the medicament delivery devices on the market today, which are set out in more detail below.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Also, when referring to e.g. the “container proximal end” and the “container distal end”, this should be construed as the proximal and distal ends of the container. The proximal and distal ends of the container may however coincide with a respective proximal end and distal end of another component. The same applies when referring to e.g. the needle cover proximal end and the needle cover distal end, as well as the below described “shield proximal end”, “shield distal end”, “cartridge proximal end” and “cartridge distal end”.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to a first aspect of the present disclosure there is provided an expulsion assembly of a medicament delivery device for expelling medicament from a medicament container structure, the expulsion assembly comprising a container holder having a container proximal end and a container distal end, the container holder being configured to house the medicament container structure, and an axially extending needle cover, the needle cover having a needle cover proximal end comprising a tubular shaped needle cover portion, and a needle cover distal end, wherein the container holder is at least partly enclosed by the needle cover, wherein a portion of the outer surface of the container holder and a portion of the needle cover form an interface section comprising an axially extending groove and a protrusion in which the container holder and the needle cover are arranged in abutment with each other.

Throughout the following disclosure, the wording “house” or “housed” should also be construed as “at least partly house”. Thus, a component defined as being housed by another component must not necessarily not be completely housed by that other component.

The tubular shaped needle cover should be construed as a portion configured to prevent exposure of a needle of the medicament delivery device. Thus, before and after usage of the medicament delivery device, the tubular shaped needle cover portion prevents a user of the medicament delivery device to accidentally be stung by the needle. According to an exemplified embodiment, the tubular shaped needle cover portion may be a cylindrical portion at the needle cover proximal end. Other shapes of the tubular shaped needle cover portion are however also conceivable, such as e.g. rectangular, quadratic, etc.

The present disclosure is based on the insight that the use of an interface section advantageously restricts the relative position between the container holder and the needle cover. In particular, the protrusion and the axially extending groove of the interface section prevents a relative rotation of the needle cover relative to the container holder, thereby providing an improved interconnection between the container holder and the needle cover. A further advantage is thus that no additional component(s) is/are necessary for sufficiently maintaining the container holder in position. Preferably, and according to an exemplified embodiment, the protrusion may be slidable along the axially extending groove. When connecting the needle cover to the container holder, the needle cover is hereby preferably slidably connected to the container holder in an axial direction thereof.

According to an exemplified embodiment, the axially extending groove may be arranged on the portion of the outer surface of the container holder, and the protrusion may be arranged on the portion of the needle cover. Hereby, the needle cover can be attached to the container holder by slidably moving, in the axial direction, the protrusion of the needle cover in the axially extending groove of the container holder.

According to an exemplified embodiment, the portion of the outer surface of the container holder may comprise a planar surface portion forming a part of the interface section. When the axially extending groove is arranged on the outer surface of the container holder, the axially extending groove is thus arranged on the planar surface portion. In a similar vein, when the protrusion is arranged on the outer surface of the container holder, the protrusion is thus arranged on the planar surface portion.

According to an exemplified embodiment, the needle cover may comprise a needle cover arm forming a part of the interface section. Preferably, and according to an exemplified embodiment, the needle cover arm may extend axially to a position distally away from the container distal end. An advantage of forming the needle cover with at least one needle cover arm is that less material is needed for the needle cover compared to a fully cylindrical needle cover.

According to an exemplified embodiment, the container holder may comprise a recess portion at the container distal end, the needle cover arm being arranged in the recess portion. The recess portion further prevents a relative rotation between the needle cover and the container holder. In detail, the needle cover arm is hereby in circumferential abutment with the recess portion of the container holder. According to an exemplified embodiment, the needle cover arm may extend distally from the tubular shaped needle cover portion.

According to an exemplified embodiment, the expulsion assembly may further comprise a needle housing member configured to house the needle of the medicament delivery device, the needle housing member being at least partly housed by the needle cover. As will be described in detail below, the needle housing member may, for example, be a removable needle shield member or a cartridge module.

According to an exemplified embodiment, one of the needle housing member and the container holder may comprise a finger portion and the other one of the needle housing member and the container holder may comprise a groove portion, the finger portion being arranged in abutment with the groove portion when the needle housing member is connected to the container holder. The abutment between the finger portion and the groove portion hereby advantageously restricts the relative motion between the needle housing member and the container holder.

According to an exemplified embodiment, the finger portion and the groove portion may be arranged in circumferential abutment with each other. An advantage is that a relative motion is substantially prevented as seen in the circumferential direction thereof. According to an exemplified embodiment, the finger portion and the groove portion may be arranged in axial abutment with each other. A still further advantage is that a relative motion, as seen in the axial direction thereof, is substantially prevented. The abutment between the finger portion and the groove portion may thus be arranged in either the circumferential direction, the axial direction, or both the circumferential direction and the axial direction.

According to an exemplified embodiment, a proximal end of the finger portion may be arranged in axial abutment with the groove portion. In this configuration, the finger portion is preferably arranged on the container holder and extends from a proximal end of the container holder.

According to an exemplified embodiment, a distal end of the finger portion may be arranged in axial abutment with the groove portion. In this configuration, the finger portion is preferably arranged on the needle housing member and extends from a distal end of the needle housing member.

As briefly indicated above, and according to an exemplified embodiment, the needle housing member may be a removable needle shield member having a shield proximal end and a shield distal end. This exemplified embodiment is particularly advantageous for a so called syringe configuration in which the needle is arranged on a medicament container structure already prior to usage of the medicament delivery device.

According to an exemplified embodiment, the needle shield member may comprise the groove portion at the shield distal end, the groove portion extending from the shield distal end in a direction towards the shield proximal end.

As also briefly indicated above, and according to an exemplified embodiment, the needle housing member may be a cartridge module having a cartridge proximal end and a cartridge distal end, the cartridge module comprising the needle at the cartridge proximal end. The cartridge module preferably comprises a needle hub holding the needle. Further exemplified details of the cartridge module will be given below in relation to the detail description of the present disclosure. This exemplified embodiment is particularly advantageous for a so called cartridge configuration in which the needle is forming part of the cartridge module. Thus, a medicament container structure for the cartridge module is not provided with a needle, which instead is arranged in the cartridge module. In further detail, the cartridge module comprises the needle, which needle is penetrating the medicament container when preparing the medicament delivery device for usage.

According to an exemplified embodiment, the cartridge module may comprise the finger portion at the cartridge distal end. The cartridge distal end should thus be construed as the distal portion of the cartridge module from which the finger portion extends. According to an exemplified embodiment, the groove portion may be a recess arranged in a surface of the container holder facing radially inwards of the container holder. The finger portion of the cartridge module can hereby attach to the groove portion. A relative motion between the cartridge module and the container holder in either of, or both, the axial and circumferential directions can hereby be substantially prevented. According to an exemplified embodiment, the recess may be a through hole.

According to an exemplified embodiment, the finger portion may comprise, at an axial end portion, a radially extending protrusion. The radially extending protrusion is thus preferably extending from the axial end portion of the finger portion. Further, the radially extending protrusion preferably extends in a radial direction relative to an axial extension of the finger portion, either radially inwards towards an axial center axis of the medicament delivery device, or in a radial direction extending away from the axial center axis of the medicament delivery device.

According to an exemplified embodiment, the radially extending protrusion may extend in a direction towards a radial center position of the expulsion assembly. This is particularly advantageous for the syringe configuration in which the needle shield member conventionally has a smaller diameter compared to the container holder. Thus, an improved interconnection between the needle shield member and the container holder is provided.

According to an exemplified embodiment, the radially extending protrusion may extend in a direction away from a radial center position of the expulsion assembly. This is particularly advantageous for the syringe configuration. Hereby, the radially extending protrusion can be snap-fitted to the groove portion.

According to a second aspect, there is provided a medicament delivery device, comprising a medicament container structure containing a medicament, and an expulsion assembly according to any one of the embodiments described above in relation to the first aspect, wherein the medicament container structure is at least partly housed within the expulsion assembly.

An advantage is, in a similar vein as described above, that the medicament delivery device is provided with an improved interconnection between the needle cover and the container holder.

According to an exemplified embodiment, the medicament delivery device may comprise a drive assembly comprising a plunger rod configured to apply an activating force on the medicament container for expelling the medicament from the medicament container structure.

The drive assembly should be construed as an arrangement which is configured to provide an activating force on the medicament container structure during use of the medicament delivery device. By means of the activating force, the medicament is expelled through a needle and into the dose delivery site. The activating force may either be generated by a manual actuation, or automatically when pushing the medicament delivery device to the dose delivery site. Automatically should here be construed such that no manual actuation is necessary. Rather, the medicament delivery device is configured in such a way that the activating force is generated by pushing the medicament delivery device to the dose delivery site. When pushing the medicament delivery device on to the dose delivery site, a motion is generated that enables actuates the activation force.

Further effects and features of the second aspect are largely analogous to those described above in relation to the first aspect.

According to a third aspect, there is provided a method of assembling an expulsion assembly of a medicament delivery device, the medicament delivery device being configured to expel a medicament from a medicament container structure, wherein the expulsion assembly comprising a container holder having a container proximal end and a container distal end, the container holder being configured to house the medicament container structure, and an axially extending needle cover, the needle cover having a needle cover proximal end comprising a tubular shaped needle cover portion, and a needle cover distal end, wherein a portion of an outer surface of the container holder and a portion of the needle cover form an interface section comprising an axially extending groove and a protrusion, wherein the method comprises the steps of inserting the container proximal end of the container holder into the needle cover at the needle cover distal end, and moving the container holder inside the needle cover until the protrusion and the axially extending groove of the interface section are arranged in abutment with each other.

Hereby, a simplified assembly method of the expulsion assembly is provided. In particular, when interface section will guide the needle cover and the container holder to a correct position. Thus, the above described method is advantageous since it reduces the risk of erroneously connecting the needle cover and the container holder to each other.

According to an exemplified embodiment, the expulsion assembly may further comprise a needle housing member configured to house the needle, wherein one of the needle housing member and the container holder comprises a finger portion and the other one of the needle housing member and the container holder comprises a groove portion, the method further comprising the steps of inserting the needle housing member into the container holder at the container proximal end, and moving the needle housing member in the distal direction until the finger portion interacts with the groove portion.

By interacting should be construed such that the finger portion and the groove portion are arranged in abutment with each other. This may be realized in a number of ways as described above in relation to the description of the first aspect. Also, the needle housing member is arranged to accommodate the needle, thus house the needle.

The needle cover and the container holder can hereby be assembled in a first stage, whereafter the needle housing member is connected to the container holder in a simple and reliable way.

Further effects and features of the third aspect are largely analogous to those described above in relation to the first and second aspects.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1A is a perspective view of a medicament delivery device in an assembled configuration according to an exemplified embodiment;

FIG. 1B is an exploded view of an expulsion assembly of the medicament delivery device in FIG. 1A according to an exemplified embodiment;

FIG. 2 is a schematic illustration of the assembly method of the expulsion assembly in FIGS. 1A-1B according to an exemplified embodiment;

FIG. 3A is an exploded view of an expulsion assembly according to another exemplified embodiment;

FIG. 3B is a perspective view of the expulsion assembly in FIG. 3A in an assembled configuration according to an exemplified embodiment;

FIG. 4 is a schematic illustration of the assembly method of the expulsion assembly in FIGS. 3A-3B according to an exemplified embodiment; and

FIG. 5 is a flow chart of a method of assembling an expulsion assembly according to an exemplified embodiment.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

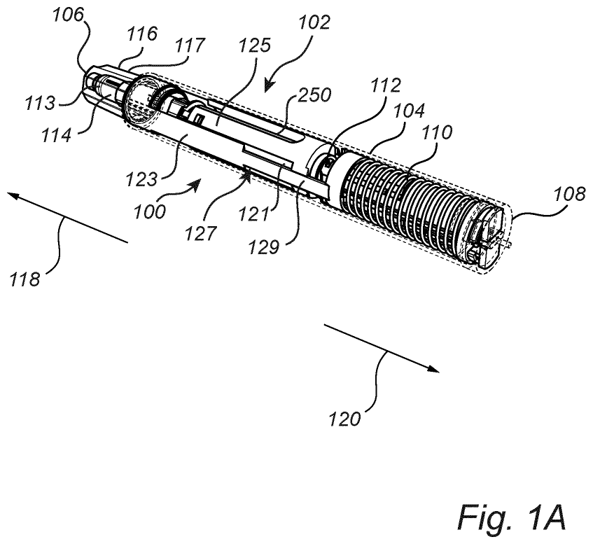

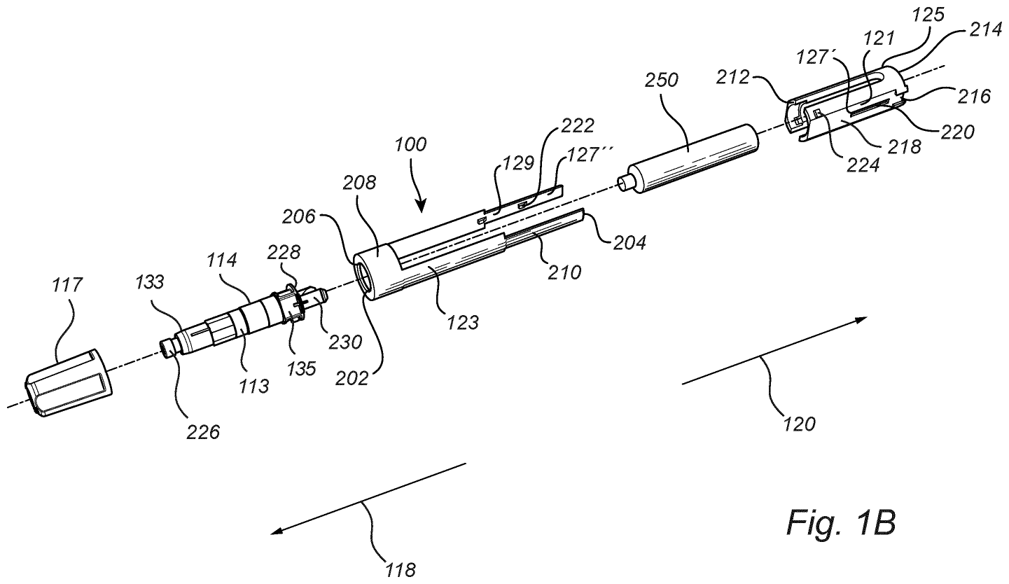

Reference is made to FIGS. 1A and 1B, which illustrate an exemplified embodiment of a medicament delivery device 102 configured to expel medicament from a medicament container structure 250. In detail, FIG. 1A illustrates the medicament delivery device 102 in an assembled configuration and FIG. 1B illustrates an expulsion assembly 100 of the medicament delivery device 102 in an exploded view according to an exemplified embodiment.

With initial reference to FIG. 1A, the medicament delivery device 102 is an axially extending medicament delivery device 102 which has a proximal end 106 and a distal end 108. The medicament delivery device 102 comprises an expulsion assembly 100. The expulsion assembly 100 comprises a container holder 125 and a needle cover 123. The container holder 125 is arranged to house the medicament container structure 250 (described in further detail below with reference to FIG. 1B), and where the container holder 125 is at least partly housed within the needle cover 123. The needle cover 123 is further arranged to prevent exposure of a needle when the medicament delivery device 102 is not in use. The needle is arranged within the below described cartridge module 114 and therefore not illustrated in the FIG. 1A embodiment.

As can be seen in FIG. 1A, a portion 121 of the container holder 125 and a portion 129 of the needle cover 123 are arranged in abutment with each other. In detail, the portion 121 of the container holder 125 and the portion 129 of the needle cover 123 form an interface section 127. As will be described below with reference to FIG. 1B, the interface section 127 comprises an axially extending groove and a protrusion arranged in abutment with each other. In yet further detail, the interface section 127 is formed by an outer surface of the container holder 125 and an inner surface of the needle cover 123 facing the container holder 125.

The medicament delivery device 102 further comprises a drive assembly 110 arranged distally from the expulsion assembly 100. The drive assembly 110 comprises a plunger rod 124 at a proximal end of the drive assembly 110. During use of the medicament delivery device 102, the plunger rod 112 is configured to apply an activating force on the medicament container structure for expelling the medicament.

Furthermore, the medicament delivery device 102 also comprises a needle housing member 113. The needle housing member 113 is configured to house the needle (not shown). The exemplified embodiment depicted in FIGS. 1A and 1B is a so-called cartridge configuration. In the cartridge configuration, the needle housing member 113 is a cartridge module 114 which comprises the needle (not shown). Thus, in the cartridge configuration, the needle is forming part of the cartridge module 114 and is not connected to the medicament container structure before usage, as is the case for the below described syringe configuration of the medicament delivery device 102. Also, the medicament delivery device 102 comprises a removable cap 116. In the FIG. 1A embodiment, the removable cap 116 is a cartridge cap 117. The needle is in the cartridge configuration penetrating the medicament container structure during use of the medicament delivery device 102.

As can also be seen in FIG. 1A, the expulsion assembly 110, the needle cover 123 and the container holder 125 are arranged within a housing 104. Also, the needle housing member 113 is at least partially arranged within the housing 104 and partly extends out of the housing in a proximal direction 118. The proximal direction 118 is thus a direction towards a dose delivery site. The distal direction 120 is thus the opposite direction compared to the proximal direction 118.

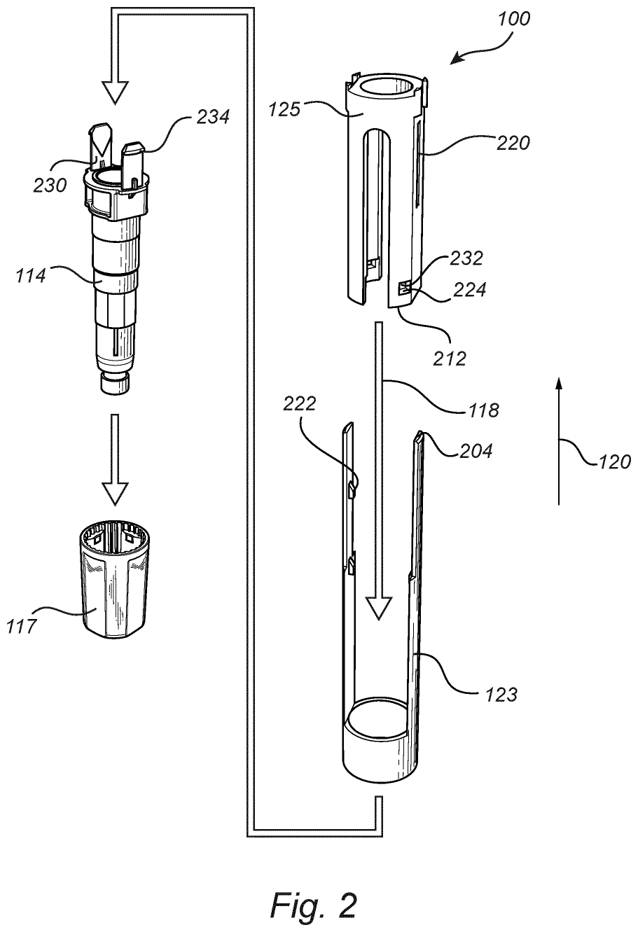

In order to describe the expulsion assembly 100 in further detail, reference is made to FIG. 1B. In particular, FIG. 1B is an exploded view of the expulsion assembly 100 in the above described cartridge configuration. As described above, the expulsion assembly 100 comprises the container holder 125, the needle cover 123 and the needle housing member 113, here in the form of a cartridge module 114, whereby a cartridge cap 117 is removably connected to the cartridge module 114. The cartridge cap 117 and the cartridge module are here defined as separate components. The cartridge module 114 should in the following be understood as a portion of the medicament delivery device 102 that comprises a needle (not shown), which will be further described below. The cartridge module 114 may preferable be formed by two or more components connected to each other forming the cartridge module 114. In particular, the cartridge module 114 comprises a cartridge proximal portion 133 and a cartridge distal portion 135, and the needle is arranged within the cartridge distal portion 135. The cartridge proximal portion 133 is removably connectable to the cartridge distal portion 135. The cartridge module 114 preferably comprises a needle hub (not shown) arranged in the cartridge proximal portion 135. The needle hub is arranged to hold the needle. Further, the needle hub is movable relative to the cartridge distal portion 135 and movable for penetrating the medicament container structure (shown in detail in FIG. 2) during use of the medicament delivery device 102. The cartridge cap 117 is a cover that is arranged at a cartridge proximal end 132 of the cartridge module 114. Thus, the cartridge cap 117 prevents exposure of the needle when attached to the cartridge module 114. Further, the cartridge cap 117 is connected to the cartridge proximal portion 133. In one example, by rotation of the cartridge cap 117 relative to the cartridge distal portion 135 around the needle, the cartridge cap 117 as well as the cartridge proximal portion 133 is removed from the cartridge distal portion 135. Hereby, the needle is exposed for usage of the medicament delivery device, i.e. for penetrating the needle at e.g. a dose delivery site. As also described above, the container holder 125 is configured to at least partly house the medicament container structure 250. The medicament container structure 250 thus contains a medicament.

The needle cover 123 has a needle cover proximal end 202 and an axially oppositely located needle cover distal end 204. At the needle cover proximal end 202, the needle cover 123 comprises a tubular shaped needle cover portion 206. According to the exemplified embodiment of FIG. 1B, the tubular shaped needle cover portion 206 is a cylindrical portion 208 arranged at the needle cover proximal end 202. The needle cover 123 also comprises a needle cover arm 210.

Preferably and as illustrated, the needle cover 123 comprises a pair of needle cover arms 210. The needle cover arm 210 extends axially in the distal direction 120 from the tubular shaped needle cover portion 206. Preferably, and as can be gleaned from FIG. 1A, the needle cover arm extends axially to a position distally away from a container distal end 214.

The container holder 125 has a container proximal end 212 and the above described, oppositely positioned container distal end 214. The container holder 125 is an axially extending component and may be arranged in e.g. a substantially cylindrical shape. At the container distal end 214, the container holder 125 comprises a recess portion 216, in which the needle cover arm 210 is positioned. The needle cover arm 210 is hereby in circumferential abutment with the recess portion 216. The recess portion 216 is arranged on a radially outer wall of the container holder 125. In one example, a surface of the recess portion 216 facing away from an axial center axis of the medicament delivery device 102. In another example, the recess can be replaced by a cut-out opening in the direction transverse to the axial center axis. The container holder 125 further comprises a planar surface portion 218 on which the needle cover arm 210 is in abutment. In one example, the planar surface portion 218 is thus arranged on the surface of the container holder 125 and has a surface facing away from the axial center axis of the medicament delivery device 102.

As described above, a portion 121 of the container holder 125 and a portion 129 of the needle cover 123 form an interface section 127. A first interface section portion 127′ of the interface section 127 is arranged at the portion 121 of the container holder 125, while a second interface section portion 127″ of the interface section 127 is arranged at the portion 129 of the needle cover 123. The interface section 127 comprises an axially extending groove 220 and a protrusion 222. The protrusion 222 is hereby slidable along the extension of the axially extending groove 220. The container holder 125 further comprises a groove portion 224 in the vicinity of the container proximal end 212. The groove portion 224 will be described in further detail below.

According to the exemplified embodiment of FIG. 1B, the axially extending groove 220 is arranged on the portion 121 of the container holder 125, i.e. forming part of the first interface section 127′, while the protrusion 222 is arranged on the portion 129 of the needle cover 123, i.e. forming part of the second interface portion 127″. The protrusion 222 is thus arranged on a surface of the needle cover arm 210 facing the container holder 125, and the axially extending groove 220 is arranged on the outer surface of the container holder 125 facing the needle cover arm 210. The protrusion 222 is here extending from the surface of the needle cover arm 210 in a direction which is perpendicular to an axial extension of the needle cover arm 210. The axially extending groove 220 is arranged as an axially extending track in the outer surface of the container holder 125, positioned between the container proximal end 212 and the container distal end 214. It should however be readily understood that the present disclosure is equally applicable with a configuration in which the axially extending groove 220 forms part of the second interface portion 127″, i.e. arranged on the needle cover 123, and in which the protrusion 222 forms part of the first interface section 127′, i.e. arranged on the container holder 125.

The cartridge module 114 has a cartridge proximal end 226 and a cartridge distal end 228, where the needle (not shown) is arranged within in the cartridge module 114 in the vicinity of the cartridge proximal end 226. The needle can be movable in the axial direction within the cartridge module 114. Further, the cartridge module 114 comprises a finger portion 230 at the cartridge distal end 228. In detail, the finger portion 230 extends axially in the distal direction 120 from the cartridge distal end 228. As will be evident from the below disclosure, the finger portion 230 is arranged to interact with the groove portion 224 of the container holder 125.

Reference is made to FIG. 2 for describing yet further details of the expulsion assembly 100 as well as describing a method of assembling the expulsion assembly 100.

During assembly of the expulsion assembly 100, the container proximal end 212 of the container holder 125 is inserted into the needle cover 123 at the needle cover distal end 204. The container holder 125 is thereafter moved relative to the needle cover 123 in the proximal direction 118 until the protrusion 222 is arranged in abutment with the axially extending groove 220. Obviously, the container holder 125 can equally as well be held stationary while moving the needle cover 123 in the distal direction 120.

The cartridge module 114 is thereafter connected to the container holder 125. As described above, the cartridge module 114 comprises a finger portion 230 and the container holder 125 comprises a groove portion 224. According to an exemplified embodiment, and as depicted in FIG. 2, the groove portion 224 is a recess portion arranged as a through hole 232 through the surface of the container holder 125. The cartridge module 114 is thus connected to the container holder 125 by arranging the finger portion 230 in abutment with the groove portion 224.

As can be seen in FIG. 2, the finger portion 230 comprises, at a distal end of the finger portion 230, a radially extending protrusion 234. The finger portion 230 thus extends from the cartridge distal end 228 and the distal end of the finger portion 230 is the position of the finger portion which is located furthest away from the cartridge distal end 228 as seen in the axial direction thereof. The radially extending protrusion 234 extends in a radial direction away from a radial center position of the expulsion assembly. Hereby, the finger portion 230 can be snap-fitted to the groove portion 224 when connecting the cartridge module 114 to the container holder 125. The radially extending protrusion 234 is thus arranged at a position of the finger portion such that it will interact with the groove portion 224 when connecting the cartridge module 114 to the container holder 125. The radially extending protrusion 234 is thus of such a shape and dimension that fits the groove portion 224. A circumferential abutment interface, as well as an axial abutment interface are hereby provided between radially extending protrusion 234 and the groove portion 224, i.e. between the finger portion 130 and the container holder 125. The cartridge cap 117 is thereafter connected to the cartridge module 114. The cartridge cap 117 can, for example, be screwed to the cartridge module 114. Other connections between the cartridge cap 117 and the cartridge module 114 are also conceivable, such as e.g. axially pushing the cartridge cap 117 onto the cartridge module 114, whereby the cartridge cap 117 is held in place by friction between a surface of the cartridge cap 117 and a surface of the cartridge module 114 interacting with each other.

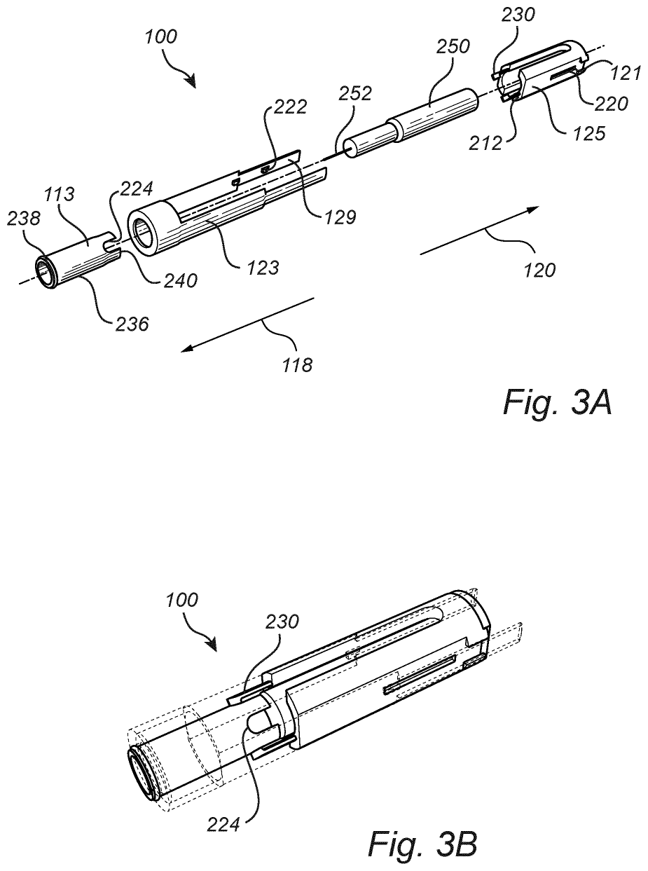

In order to describe the syringe configuration of the expulsion assembly 100, reference is made to FIGS. 3A and 3B. In particular, FIG. 3A is an exploded view of the expulsion assembly 100 in the syringe configuration and FIG. 3B illustrates the syringe configuration in an assembled configuration. The syringe configuration comprises many features similar to those described above in relation to FIGS. 1A-2, which similar features will not be described in detail unless indicated.

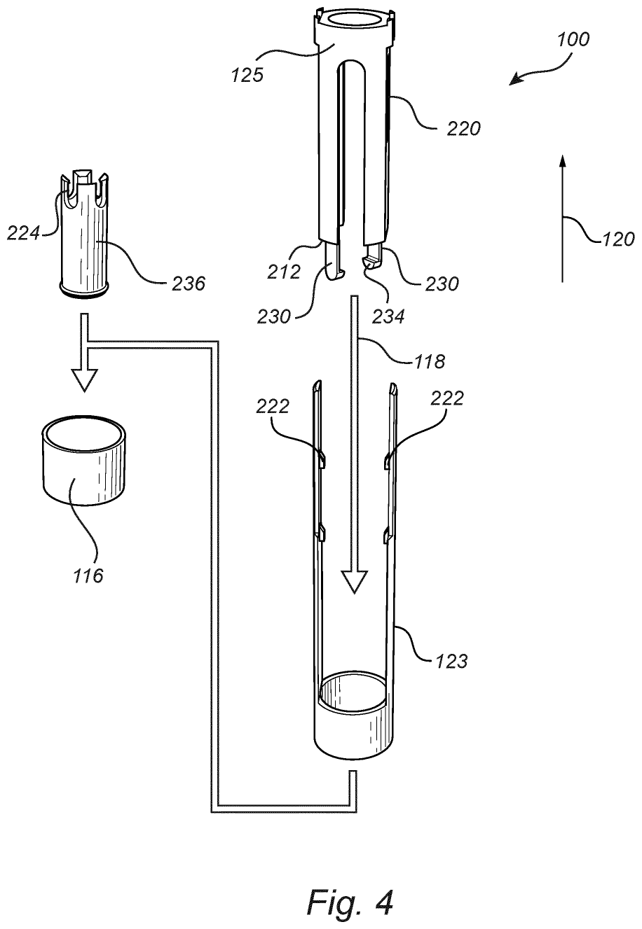

The expulsion assembly 100 in FIGS. 3A-3B comprises, in a similar vein as the embodiment in FIGS. 1A-2, a container holder 125 arranged to house the medicament container structure 250, a needle cover 123 and a needle housing member 113. In the syringe configuration, the needle 252 is arranged on the medicament container structure 250, i.e. the needle 252 forms a part of the medicament container structure 250, in comparison to the needle, before usage of the medicament delivery device 102, forms part of the cartridge module 114 for the cartridge configuration described above. The needle 252 may preferably be protected by use of a rigid needle shield (not shown). The rigid needle shield is an axially extending portion arranged to embed the needle 252 and protect the needle 252 from external damage. The expulsion assembly 100 also preferably comprises a removable cap 116 (FIG. 4).

In a similar vein as the cartridge configuration, a portion 121 of the container holder 125 and a portion 129 of the needle cover 123 form an interface section 127. As can be seen in FIG. 3A, the portion 121 of the container holder 125 comprises the axially extending groove 220 and the portion 129 of the needle cover 123 comprises the protrusion 222.

The needle housing member 113 of the embodiment depicted in FIGS. 3A-3B is a removable shield member 236 having a shield proximal end 238 and a shield distal end 240. The removable shield member 236 is configured to house the needle 252 as well as the above described rigid needle shield. The removable shield member 236 is dimensioned to fit into the needle cover 123 at the needle cover proximal end 202. In the exemplified embodiment of FIGS. 3A-3B, the removable shield member 236 is arranged as a cylindrical tube portion.

A further difference between the embodiment depicted in FIGS. 1A-2, and the embodiment depicted in FIGS. 3A-3B, is that the above described groove portion 224 is arranged as a portion on the needle housing member 113, i.e. a portion on the removable shield member 236, while the above described finger portion 230 forms part of the container holder 125. In yet further detail, the groove portion 224 is arranged at the shield distal end 240 and extends from the shield distal end 240 in the proximal direction 118, i.e. towards the shield proximal end 238. The finger portion 230 on the other hand is arranged at the container proximal end 212 and extends in the proximal direction 118. In the assembled configuration depicted in FIG. 3B, the finger portion 230 is arranged in abutment with the groove portion 224. In detail, the finger portion 230 is arranged in axial abutment as well as in circumferential abutment with the groove portion 224. A proximal end of the finger portion 230 is thus arranged in abutment with a proximal end of the groove portion 224, while side wall portions of the finger portion 230 prevents a relative rotation between the removable shield member 236 and the container holder 125.

Reference is made to FIG. 4 for describing yet further details of the expulsion assembly 100 of the FIGS. 3A-3B embodiment, as well as describing a method of assembling the expulsion assembly 100.

In a similar vein as for the embodiment described above in relation to FIG. 2, the expulsion assembly depicted in FIG. 4 is assembled by inserting the container proximal end 212 of the container holder 125 into the needle cover 123 at the needle cover distal end 204. The container holder 125 is thereafter moved relative to the needle cover 123 in the proximal direction 118 until the protrusion 222 is arranged in abutment with the axially extending groove 220. Obviously, the container holder 125 can equally as well be held stationary while moving the needle cover 123 in the distal direction 120.

The removable needle shield member 236 is thereafter connected to the container holder 125. As described above, the removable needle shield member 236 comprises a groove portion 224 and the container holder 125 comprises a finger portion 230. As can be seen in FIG. 4, the finger portion 230 comprises, at a proximal end of the finger portion 230, a radially extending protrusion 234. The finger portion 230 thus extends from the container proximal end 212 and the proximal end of the finger portion 230 is the position of the finger portion which is located furthest away from the container proximal end 212 as seen in the axial direction thereof. The radially extending protrusion 234 extends in a radial direction towards a radial center position of the expulsion assembly. Since the diameter of the removable needle shield member 236 is smaller than the distance between the finger portions 230, the radially extending protrusion 234 can be arranged in abutment with the groove portion(s) 224. The removable cap is thereafter connected to the removable needle shield member 236.

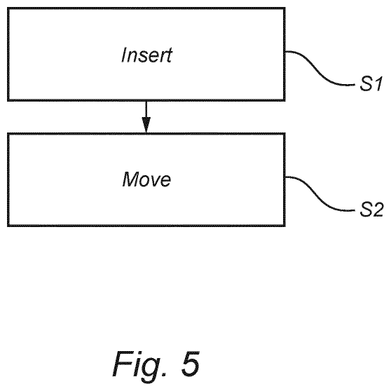

In order to sum up, reference is made to FIG. 5 which is a flow chart of a method of assembling the expulsion assembly described above in relation to FIGS. 1-4. As has been described, the method is initiated by inserting S1 the container proximal end 212 of the container holder 125 into the needle cover 123 at the needle cover distal end 204. The container holder 125 is thereafter moved S2 relative to the needle cover 123 in the proximal direction 118. The container holder 125 is moved inside the needle cover 123 until the protrusion 222 and the axially extending groove 220 are arranged in abutment with each other.

The method thereafter preferably further comprises the steps of inserting the needle housing member 113, i.e. the cartridge module 114 or the removable needle shield member 236, into the container holder 125 at the container proximal end 212, and moving the needle housing member 113 in the distal direction 120 until the finger portion 230 interacts with the groove portion 224.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. An expulsion assembly of a medicament delivery device for expelling medicament from a medicament container structure, the expulsion assembly comprising:
a container holder having a container proximal end and a container distal end, the container holder being configured to house the medicament container structure; and
an axially extending needle cover, the needle cover having a needle cover proximal end comprising a tubular shaped needle cover portion, and a needle cover distal end, wherein the container holder is at least partly enclosed by the needle cover,
wherein a portion of an outer surface of the container holder and a portion of the needle cover form an interface section comprising an axially extending groove and a protrusion in which the container holder and the needle cover are arranged in abutment with each other.

17. The expulsion assembly according to claim 16, wherein the protrusion is slidable along the axially extending groove.

18. The expulsion assembly according to claim 16, wherein the axially extending groove is arranged on the portion of the outer surface of the container holder, and the protrusion is arranged on the portion of the needle cover.

19. The expulsion assembly according to claim 16, wherein the needle cover comprises a needle cover arm forming a part of the interface section.

20. The expulsion assembly according to claim 16, wherein the expulsion assembly further comprises a needle housing member configured to house the needle, the needle housing member being at least partly housed by the needle cover.

21. The expulsion assembly (100) according to claim 20, wherein one of the needle housing member and the container holder comprises a finger portion and the other one of the needle housing member and the container holder comprises a groove portion, the finger portion being arranged in abutment with the groove portion when the needle housing member is connected to the container holder.

22. The expulsion assembly according to claim 21, wherein the finger portion and the groove portion are arranged in circumferential abutment with each other.

23. The expulsion assembly according to claim 21, wherein the finger portion and the groove portion are arranged in axial abutment with each other.

24. The expulsion assembly according to claim 20, wherein the needle housing member is a removable needle shield member having a shield proximal end and a shield distal end.

25. The expulsion assembly according to claim 24, wherein the needle shield member comprises the groove portion at the shield distal end, the groove portion extending from the shield distal end in a direction towards the shield proximal end.

26. The expulsion assembly according to claim 20, wherein the needle housing member is a cartridge module having a cartridge proximal end and a cartridge distal end, the cartridge module comprising the needle at the cartridge proximal end.

27. The expulsion assembly according to claim 26, wherein the cartridge module comprises the finger portion at the cartridge distal end.

28. A medicament delivery device, comprising
a medicament container structure containing a medicament, and
an expulsion assembly according to claim 16, wherein the medicament container structure is at least partly housed within the expulsion assembly.

29. A method of assembling an expulsion assembly of a medicament delivery device, the medicament delivery device being configured to expel a medicament from a medicament container structure, wherein the expulsion assembly comprises a container holder having a container proximal end and a container distal end, the container holder being configured to house the medicament container structure, and an axially extending needle cover, the needle cover having a needle cover proximal end comprising a tubular shaped needle cover portion, and a needle cover distal end, wherein a portion of an outer surface of the container holder and a portion of the needle cover form an interface section comprising an axially extending groove and a protrusion, wherein the method comprises the steps of:
inserting the container proximal end of the container holder into the needle cover at the needle cover distal end; and
moving the container holder inside the needle cover until the protrusion and the axially extending groove of the interface section are arranged in abutment with each other.

30. The method according to claim 29, wherein the expulsion assembly further comprises a needle housing member configured to house the needle, wherein one of the needle housing member and the container holder comprises a finger portion and the other one of the needle housing member and the container holder comprises a groove portion, the method further comprising the steps of:
inserting the needle housing member into the container holder at the container proximal end; and
moving the needle housing member in the distal direction until the finger portion interacts with the groove portion.
